(19) United States
(12) Patent Application Publication
(10) Pub. No.: US 2016/0077084 A1
(43) Pub. Date: Mar. 17, 2016

(54) AMPHIBIAN OOCYTE OR EMBRYO BIOASSAY

(71) Applicants: Narsimha Reddy PENTHALA, Little Rock, AR (US); Peter CROOKS, Little Rock, AR (US); Venumadhav JANGANATI, Little Rock, AR (US); Angus MacNicol, Little Rock, AR (US); THE BOARD OF TRUSTEES OF THE UNIVERSITY OF ARKANSAS, Little Rock, AR (US)

(72) Inventors: Angus MacNicol, Little Rock, AR (US); Narsimha Reddy Penthala, Little Rock, AR (US); Peter Crooks, Little Rock, AR (US); Venumadhav Janganati, Little Rock, AR (US)

(21) Appl. No.: 14/785,196

(22) PCT Filed: Apr. 18, 2014
(86) PCT No.: PCT/US14/34604

§ 371 Date: Oct. 16, 2015

Related U.S. Application Data
(60) Provisional application No. 61/814,020, filed on Apr. 19, 2013.
(60) Provisional application No. 61/866,872, filed on Aug. 16, 2013.
(60) Provisional application No. 61/871,657, filed on Aug. 29, 2013.

(51) Int. Cl. G01N 33/50

(52) U.S. Cl. CPC G01N 33/5088

(57) ABSTRACT

The present invention provides in vivo methods for screening compounds of interest. The methods rely on readily-observable phenotypic changes in amphibian oocytes or early embryos.

### CROSS-REFERENCE TO RELATED APPLICATIONS

This application claims the benefit of PCT Application PCT/US2014/034604, filed Apr. 18, 2014, which claims the benefit of U.S. Provisional Patent Application No. 61/814,020, filed Apr. 19, 2013, U.S. Provisional Patent Application No. 61/866,872, filed Aug. 16, 2013, and U.S. Provisional Patent Application No. 61/871,657 filed Aug. 29, 2013, which are each hereby incorporated by reference in their entirety.

### GOVERNMENTAL RIGHTS

This invention was made with government support under HD035688 and CA158275I awarded by the National Institutes of Health. The government has certain rights in the invention.

### FIELD

The present invention relates to a phenotypic assay for screening compounds of interest.

### BACKGROUND

Forward chemical genetic screens use small molecules to change the way proteins work by interacting directly in real time, rather than indirectly by manipulating their genes. Such screens can be used to identify which proteins regulate different biological processes, to understand the molecular basis of their biological functions, and to identify small molecules that may be of medical value. Screening chemical compounds involves complex repetitive assays for biochemical or cellular endpoints involving both initial hit identification and subsequent validation. Mammalian models of drug tolerance, dose, uptake, and elimination are expensive and require large amounts of test compound. Thus, there is a need for relatively rapid, cost-effective alternates to mammalian models, at least for the initial screening steps, to reduce the time and cost burden of new drug discovery.

Given the extensive literature on early developmental pathways and their mechanisms of action, amphibian oocytes and embryos offer a powerful tool for probing pathway function with small molecules and subsequently leveraging the well characterized biochemical and molecular assays to pinpoint the cellular target of the candidate small molecule. Additionally, amphibian oocytes and early embryos provide unprecedented access to translational regulatory mechanisms because early development occurs in the absence of gene transcription. Rather, the early cell cycles and developmental processes are regulated by proteins that are synthesized from pre-existing, maternally-inherited mRNAs in a specific temporal pattern that is controlled through sequence-specific mRNA binding proteins. It is well known that regulated mRNA translation plays a key role in controlling cell growth and cell survival, and, as such, is an important therapeutic target for cancer control. Thus, amphibian oocytes and embryos provide an in vivo screening method in which readily observable phenotypic changes can be used to identify compounds with potential therapeutic value.

### SUMMARY

Among the various aspects of the present disclosure is the provision of an in vivo method for screening a plurality of compounds. The method comprises contacting a plurality of amphibian oocytes or embryos with the plurality of compounds, and monitoring a phenotype in the plurality of amphibian oocytes or embryos to identify a compound that affects the phenotype.

Another aspect of the present disclosure provides a method for identifying a compound that affects a regulated mRNA translation control process. The contacting a plurality of amphibian oocytes or embryos with a plurality of compounds, and monitoring a phenotype in the plurality of amphibian oocytes or embryos to identify the compound that affects the regulated mRNA translational control process.

Other aspects and iterations of the disclosure are described in more detail below.

### BRIEF DESCRIPTION OF THE DRAWINGS

The application file contains at least one photograph executed in color. Copies of this patent application publication with color photographs will be provided by the Office upon request and payment of the necessary fee.

FIG. 1 presents a schematic of the oocyte and early embryo phenotypic screening assays. Hit validation indicates confirmation of meiotic or mitotic cell cycle perturbation at the original test dose as well as analysis of serial dilutions of candidate compounds.

FIG. 2 depicts a proposed sequential hierarchy of mRNA translational control factors and signal transduction pathways that govern maternal mRNA activation at specific phases in the cell cycle in amphibian oocytes.

FIG. 3 shows a Western blot illustrating that MAP kinase activation can be detected by phospho-specific antisera several hours prior to GVBD50 (at 5 hours in this experiment).

FIG. 4A, FIG. 4B, FIG. 4C, FIG. 4D and FIG. 4E present images of Xenopus oocytes cultured in a 96-well plate. Immature stage VI oocytes were cultured overnight in the presence of 1% DMSO. Oocyte maturation was stimulated by the addition of progesterone. The 96-well plate was photographed using a Nikon D800 36 megapixel SLR camera. FIG. 4A, FIG. 4B, FIG. 4D show digitally zoomed images of the indicated wells of the plate shown in FIG. 4E. FIG. 4A shows oocytes incubated with progesterone that had undergone germinal vesicle breakdown (GVBD) (arrowheads indicate oocytes that had not yet completed GVBD). FIG. 4B shows immature oocytes that were incubated without progesterone. FIG. 4C and FIG. 4D show peripheral wells containing oocytes incubated with and without progesterone, respectively. Although some minor distortion was observed in these wells, the entire field of oocytes was observable.

FIG. 5 illustrates hormone-independent activation of maturation by compound PNR-5-46. Presented are digital capture images of treated oocytes in a multiwell format. Images represent same-well optical zoom from time-lapse images. White spots on the dark animal hemispheres are clearly visible and are a measure of oocyte GVBD and maturation rate. Upper panel, oocytes treated with DMSO (1% v/v final). Middle panel, oocytes treated with 100 μM PNR-5-46. Lower panel, oocytes treated with progesterone. Rate of progression to GVBD is indicated below each image as the proportion of total oocytes in each well.

FIG. 6 illustrates the activation of maturation by compound JVM-9 in the absence of progesterone. Plotted is the percent of oocytes at GVBD at the indicated time points over a range of concentrations of JVM-9 (labeled 6e) relative to DMSO-treated control oocytes.

FIG. 7 shows the attenuation of progesterone-induced GVBD by compound PNR-5-41. Plotted is the percent of oocytes at GVBD at the indicated time points over a range of concentrations of PNR-5-41.

FIG. 8A and FIG. 8B show the effect of activators on the phosphorylation status of MAP kinase (MAPK), Cdc2, and Musashi2 (Msi2). FIG. 8A presents Western blots of oocytes incubated with or without PNR-5-46 (5 μM) or JVM-9 (100 μM); each blot was probed with the indicated phospho-specific antibody or an anti-tubulin antibody (as a control). FIG. 8B shows Western blots of oocytes incubated with PNR-5-46, two other activators, or DMSO; blots were probed with the indicated antibodies (GAPDH is a control).

FIG. 9 illustrates the effect of inhibitor PNR-5-41 on the phosphorylation status of Musashi1 (Msi1), MAP kinase (MAPK), and Cdc2 (CDK1). Shown are Western blots of oocytes incubated with or without vehicle (DMSO) or PNR-5-41; each blot was probed with the indicated phospho-specific antibody.

FIG. 10A and FIG. 10B show inhibition of breast cancer cell self-renewal by parthenolide derivatives. Plotted is the percent of mammosphere-forming units (MFUs) at day 14 (P2) per cells plated (at day 7, P1) for cells treated with each of the indicated compounds (until day 3). FIG. 10A shows MCF-7 cells. FIG. 10B shows MDA-MB-231 cells. Values with different letters (a, b, c) differed at P<0.05. N=3 experiments, carried out in quadruplicate.

FIG. 11A and FIG. 11B present inhibition of neural cancer cell self-renewal by parthenolide derivatives. Plotted is the percent of sphere formation relative to DMSO-treated cells for the indicated compounds at the indicated doses. FIG. 11A shows SHSY5Y 7 cells. FIG. 11B presents U87 cells. * P<0.05; ** P<0.01; *** P<0.001 by ANOVA. N=3 experiments, carried out in quadruplicate.

FIG. 12A and FIG. 12B present blastula stage Xenopus embryos. FIG. 12A shows a control blastula stage embryo derived from a one cell embryo that was sham injected. FIG. 12B shows presents an embryo in which cell divisions were perturbed following microinjection of vRaf mRNA at the one cell stage.

### DETAILED DESCRIPTION

Provided herein are methods for using amphibian oocytes or embryos to screen compounds of interest. Cellular and molecular events during amphibian oocyte maturation and early embryogenesis have been extensively studied and well-characterized. Because amphibian oocytes or embryos provide readily-observable and rapid phenotypic changes, they offer an attractive in vivo platform for both novel drug discovery and target identification. Thus, the disclosed amphibian oocyte/embryo bioassay provides a time-efficient and cost-effective screening alternative to mammalian cell-based or biochemical screening systems. Because of the extensively-characterized mechanisms underlying oocyte maturation and early embryogenesis, the disclosed in vivo screening methods provide the opportunity to rapidly establish the mechanism of action of a compound of interest, biochemically identify the compound's target, and give an initial indication of possible toxicity issues. Additionally, because gene transcription is suppressed during amphibian oocyte maturation and early embryonic development, the disclosed bioassay provides a rapid and powerful system to identify compounds that affect mRNA translation control processes.

### I. In Vivo Screening Methods

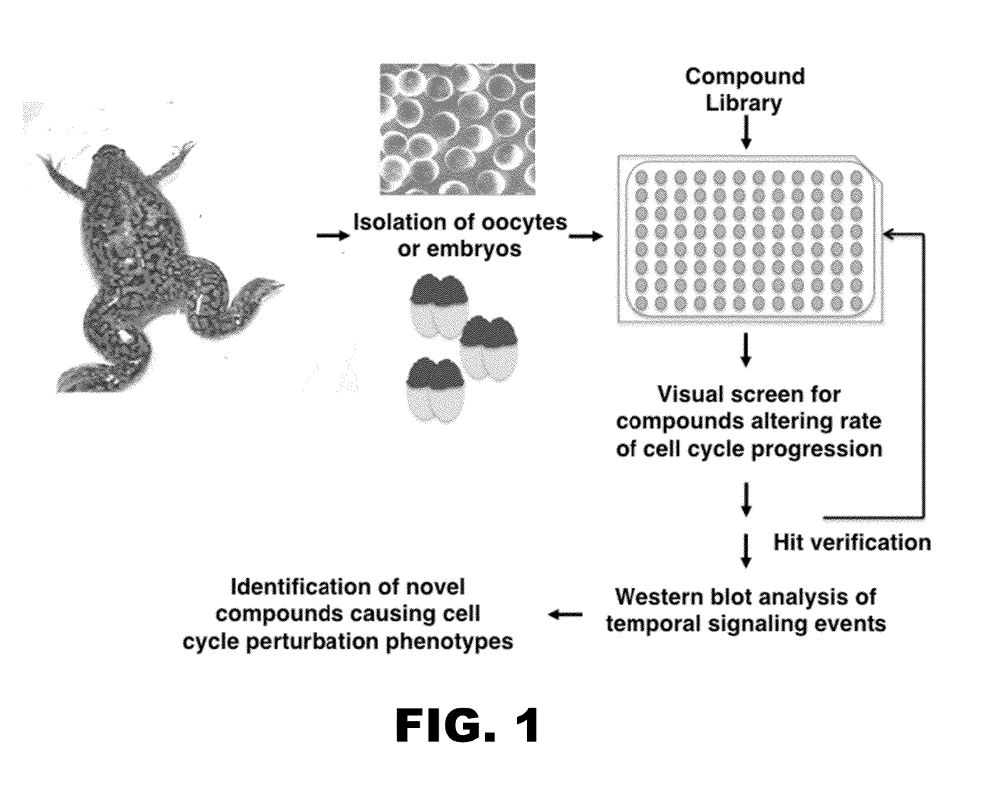

One aspect of the present disclosure encompasses in vivo methods for screening a plurality of compounds. The methods comprise a) contacting a plurality of amphibian oocytes or embryos with a plurality of compounds, and b) monitoring a phenotype in the plurality of amphibian oocytes or embryos to identify a compound that affects the phenotype. A schematic overview of the screening process is shown in FIG. 1.

Amphibian oocytes and early embryos are transcriptionally silent and rely on large stores of maternally-produced mRNAs. The proteins required for cell cycle progression and early developmental processes are synthesized from the pre-existing pool of maternally-produced mRNAs in a specific temporal process that is predominately controlled through sequence-specific mRNA binding proteins. The disclosed screening processes, therefore, may identify compounds that target proteins involved in processes controlled by regulated mRNA translation. Specifically, compounds may be identified that affect conserved mRNA translational control proteins, cell cycle control proteins, and/or signal transduction pathway proteins.

### (a) Contacting

The screening methods disclosed herein comprise contacting a plurality of amphibian oocytes or embryos with a plurality of compounds.

(i) Amphibian Oocytes and Embryos

A variety of amphibian oocytes or embryos may be used in the screening methods disclosed herein. Large numbers of amphibian oocytes are readily available from adult females. Immature oocytes can be stimulated to mature in vitro, mature oocytes can be fertilized in vitro, and the oocytes and embryos are easily cultured in vitro in simple salt solutions. Additionally amphibian oocytes or embryos tend to be quite large, making them easy to manipulate experimentally. In one embodiment, the amphibian oocytes or embryos used in the screening methods are from the subclass Lissamphibia, which includes frogs, toads, salamanders, mudpuppies, newts, and caecilians. In some embodiments, the amphibian oocytes or embryos are frog oocytes or embryos. Exemplary frogs include species of Xenopus and Rana. In some embodiments, the amphibian oocytes or embryos may be Rana pipiens. In other embodiments, the amphibian oocytes or embryos may be Xenopus tropicalis. In still other embodiments, the amphibian oocytes or embryos may be Xenopus laevis.

In general, the oocytes used in the screening methods are immature oocytes. The immature oocytes may be stage V or stage VI oocytes. In exemplary embodiments, the oocytes may be stage VI oocytes, which are arrested in late G2 phase of meiosis, just prior to meiotic entry. Typically, the embryos used in the method are pre-blastula (i.e., cleavage) stage embryos. Suitable pre-blastula stage embryos include stage 1 (fertilized 1-cell), stage 2 (2-cell), stage 3 (4-cell), stage 4 (8-cell), stage 5 (16-cell), and stage 6 (32-cell) embryos. In other embodiments, the embryos may be later stage embryos (i.e., later than stage 6).

The amphibian oocytes or embryos used in the screening methods may be wild type. Alternatively, the amphibian oocytes or embryos used in the screening methods may be mutant or derived from a mutant female. The mutant amphibian may be naturally-occurring or genetically-modified. For example, a genetically-modified amphibian may comprise at least one exogenous nucleic acid encoding a protein of interest. The exogenous sequence may be chromosomally-integrated or it may be extrachromosomal. The encoded protein of interest may be a reporter protein such as, for example, a green fluorescent protein (GFP), red fluorescent protein (RFP), or another fluorescent protein. In other instances, the encoded protein of interest may be a selectable marker protein. In still of instances, the encoded protein of interest may be a fusion protein comprising a marker domain. Non-limiting examples of suitable marker domains include fluorescent proteins, glutathione-S-transferase (GST), chitin binding protein, maltose binding protein, and epitope tags such as 6xHism Myc, FLAG, HA and the like. In additional embodiments, the genetically-modified amphibian may comprise an inactivated (i.e., knocked-out) or modified protein-coding gene. Target genes that may be inactivated or modified include, but are not limited to, Pumilio, Musashi1, Musashi2, CPEB (i.e., cytoplasmic polyadenylation element binding) protein, Ringo, Cyclin A1, Cyclin B1, Cyclin B2, Cyclin B4, Cyclin B5, Cdk1, Cdk2, Cdc25, Wee1, MAPK, Mos, MEK1, Rsk1/2, Myt1, P13K, and other signaling proteins. Genetically-modified amphibians may be generated using well-known techniques such as homologous recombination, transposon-mediated insertion, and targeted modifications using zinc finger nucleases (ZFNs), meganucleases, or transcription activator-like effector (TALE) nucleases.

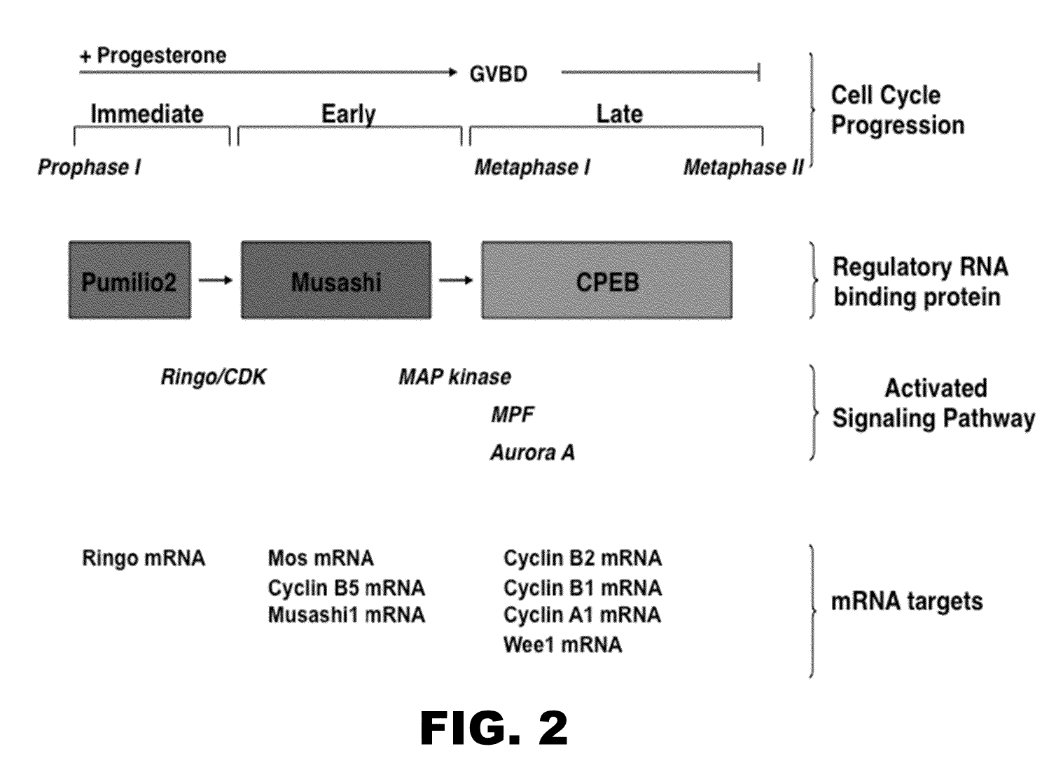

Oocyte maturation in frogs and other amphibians generally is triggered by a hormone and comprises breakdown of the germinal vesicle (i.e., oocyte nucleus) indicating entry into metaphase of meiosis (see FIG. 2). Oocyte cell cycle progression is controlled by proteins synthesized from pre-existing (i.e., inherited) maternally-produced mRNAs. These proteins are synthesized in a specific temporal pattern that is predominantly controlled through sequence-specific mRNA binding proteins, including, without limit, the developmental regulator, Pumilio, the stem cell self-renewal factor, Musashi, and the cytoplasmic polyadenylation binding protein, CPEB. More specifically, hormone stimulation leads to inhibition of Pumilio function causing de-repression and translation of Ringo mRNA. Ringo protein activates cyclin-dependent kinase (CDK) which phosphorylates and activates Musashi to promote early mRNA translation. Musashi-dependent translation of the early class Mos mRNA results in MAP kinase activation and subsequent CPEB-mediated, late class mRNA translation. The sequential function of Pumilio, Musashi- and CPEB-dependent translational control promotes and maintains M-phase promoting factor activity (MPF, which is a cyclin B/CDK 1 complex) and cell cycle progression up to metaphase of Meiosis II. Upon fertilization, the embryo undergoes a series of rapid synchronous cleavage divisions that divide the embryo into smaller and smaller cells. The early embryonic cell cycles are also controlled by proteins synthesized from pre-existing maternally-produced mRNAs. The embryo undergoes a transition from maternal to zygotic transcription during the midblastula stage (this transition is termed the midblastula transition or MBT).

For the in vivo screening methods, the plurality of amphibian oocytes or embryos may be disposed in a multi-well system. The use of multi-well systems allows for high-throughput screening formats, wherein multichannel pipette systems, robotic liquid handling systems, automated detection devices, etc. may be used to quickly screen many thousands of compounds of interest. The multi-well system may be a plate, a dish, or a slide; and the multi-well system may comprise polystyrene, polycarbonate, polypropylene, glass, silica, or metal. The wells of the multi-well system may have flat bottoms or round bottoms. The wells may be surface-coated or culture-treated. In exemplary embodiments, the multi-well system is a multi-well plate. The multi-well plate may be 6-well, 12-well, 24-well, 48-well, 96-well, 384-well, and so forth.

Each well of the multi-well system comprises at least one oocyte or embryo for the screening method. In various embodiments, each well of the multi-well system may contain from 1-5, 6-10, 11-15, 16-20, 21-25, 26-30, 31-35, 36-40, 41-45, 46-50, 51-55, 56-60, 61-70, 71-80, 81-90, or more than 90 oocytes or embryos. In one embodiment, the multi-well system may be a 96-well plate and each well may contain 20 or 21 oocytes or embryos. In another embodiment, the multi-well system may be a 96-well plate and each well may contain 2-5 oocytes or embryos. The oocytes or embryos may be distributed using micropipettes or robotic handling systems.

Each well of the multi-well system also comprises a suitable medium for incubation of the oocytes or embryos. Suitable media include L-15 medium (Leibovitz), modified L-15 medium, a buffered saline solution, Barth's saline, modified Barth's saline, E2 embryo medium, E3 embryo medium, and the like. The medium may be distributed to the wells of the multi-well system using multichannel pipette systems or robotic liquid handling systems.

(ii) Plurality of Compounds

The method comprises contacting the plurality of amphibian oocytes or embryos with a plurality of compounds. The plurality of compounds may be a library of compounds. Suitable compounds include small molecules, pharmaceutically active compounds (i.e., drugs), natural products, carbohydrates, lipid molecules, amino acid derivatives, peptides, peptide mimetics, nucleic acids, antisense oligonucleotides, microRNAs, and so forth. In exemplary embodiments, the plurality of compounds is a library of small molecules. In general, a small molecule is defined as a molecule having a molecular weight of less than about 1000 daltons (Da). In other embodiments, the plurality of compounds may comprise larger molecules that are cell permeable.

Libraries of small molecules are available through repositories or commercial sources, and means for generating libraries of small molecules are well known in the art. Exemplary small molecule compounds include those that may affect RNA binding proteins, translational control proteins, proteins involved in RNA masking and/or unmasking, cell cycle control proteins, replication control proteins, chromatin remodeling proteins, mitotic control proteins, cell division control proteins, proteins involved in nuclear membrane assembly or disassembly, signal transduction pathways such as MAPK, Wnt, Notch, Hedgehog, etc., membrane receptors, receptor tyrosine kinases, intracellular kinases, phosphatases, and other enzymes.

In some embodiments, the compounds to be screened may comprise a tag. Suitable tags include biotin, fluorophores, dyes, fluorocarbon tags, click chemistry tags, affinity tags, and the like. Biotin is an exemplary tag. The presence of the tag may permit isolation of a complex comprising the compound and a cellular target with which the compound interacts.

The compounds to be screened generally will be dissolved in a suitable solvent (such as, e.g., DMSO or ethanol) and added to the medium containing the oocytes or embryos. The plurality of compounds may be distributed to the wells of a multi-well system using multichannel pipette systems or robotic liquid handling systems. Initially, the oocytes or embryos will be contacted with a single concentration of each compound. If the initial concentration of a compound is toxic to the oocytes or embryos, then that compound will be retested at a lower concentration. Additionally, compounds that affect the phenotype that is monitored will be rescreened at several different concentrations to determine the half maximal effective concentration (EC₅₀). For statistical purposes, contact with a compound of interest will be performed in at least duplicate or triplicate.

During each screening procedure, a small percentage of oocytes or embryos will serve as untreated controls. That is, the untreated oocytes or embryos will not be contacted with any compounds of interest but rather will be contacted only with the solvent used to dissolve the compounds of interest and/or the hormone used to stimulate oocyte maturation, as appropriate.

The temperature of the contacting step can and will vary, depending, for example, upon the species of the oocytes or embryos. In some embodiments, the temperature of the contacting step may range from about 10-15° C., 15-20° C., 20-23° C., 23-25° C., 25-30° C., or 30-35° C. In certain embodiments the temperature of the contacting step may range from about 16-25° C.

In embodiments in which oocytes are utilized in the screening method, the oocytes may be preincubated with the compounds of interest for a period of time to permit entry of the compound into the oocytes. The preincubation period may range from several hours to several days. In various embodiments, the preincubation period of time of may be about 1, 2, 3, 4, 5, 6, 7, 8, 9, 10, 11, 12, 13, 14, 15, 16, 17, 18, 19, 20, 21, 22, 23, 24 hours, or longer. After the preincubation period, the oocytes may be contacted with an effective concentration of a suitable hormone to stimulate oocyte maturation such that a phenotype can be observed. Non-limiting examples of hormones that stimulate oocyte maturation include progesterone, insulin, and insulin-like growth factors. In other embodiments, the compounds of interest may stimulate oocyte maturation in the absence of hormone stimulation. In such cases, monitoring of the phenotype will begin soon after addition of the compounds of interest.

In embodiments in which embryos are used, phenotype monitoring generally will begin soon after contact between the compounds of interest and the fertilized embryos. Thus, there generally will not be sufficient time for a preincubation period prior to phenotype monitoring, and compounds that readily do not cross the cell membrane may not be adequately screened. In other embodiments in which embryos are used, it may be possible to preincubate unfertilized (mature) oocytes with the compounds of interest and then fertilize the oocytes in each well of a multi-well system at which time monitoring of the early embryonic phenotype can begin. In such cases, the compounds of interest may have sufficient time to cross the oocyte cell membrane, but fertilization may be less efficient.

### (b) Monitoring

The in vivo screening method further comprises monitoring a phenotype in the plurality of oocytes or embryos such that compounds that affect the phenotype can be identified. The phenotype may be controlled, directly or indirectly, by a regulated mRNA translation control process that involves a RNA translation control protein. Non-limiting examples of RNA translation control proteins include Pumilio1, Pumilio2, Musashi1, Musashi2, or the cytoplasmic polyadenylation element binding protein (CPEB).

(i) Phenotypes

In some embodiments, the phenotype may be a visually observable phenotype. For example, the visually observable phenotype may be germinal vesicle breakdown (GVBD), which is an indicator of oocyte maturation. GVBD is observable as white spot or clearing at the darkly pigmented animal pole of the oocyte. The white spot or clearing is due to the migration of the germinal vesicle to the animal pole immediately prior to its breakdown. Typically, GVBD occurs about 3-10 hours after hormone-dependent stimulation of oocyte maturation. In some embodiments, the compounds of interest may affect the kinetics of hormone-stimulated GVBD (e.g., the timing of hormone-induced GVBD may be accelerated or inhibited). In other embodiments, the compounds of interest may activate GVBD in the absence of hormone stimulation. In further embodiments, the compound of interest may inhibit GVBD after hormone exposure. The timing of GVBD may be expressed percentage of oocytes to undergo GVBD at a specific time. Alternatively, times may be standardized between experiments as the time taken for a certain percent (e.g., 50%) of the oocytes to undergo GVBD.

In other embodiments, the visually observable phenotype may comprise early embryonic cell cleavage divisions. Fertilized one-cell amphibian embryos undergo a stereotyped series of cleavages in which the embryo is divided into increasingly smaller cells. In Xenopus embryos, for example, the first cleavage occurs about 90 minutes after fertilization, and the next 11 rounds of cleavage occur at about 20-30 minute intervals. The compounds of interest may affect the timing of cell division. Alternatively, the compounds of interest may affect the symmetry or spatial orientation of cell division.

In still other embodiments, the phenotype may be a reporter-based assay. For example, the oocytes or embryos used in the screening method may comprise a fluorescent protein, a fusion protein comprising a marker domain, or another reporter molecule which can be used to monitor the activity of a protein of interest, a molecular event of interest, or a cell signaling event of interest.

In alternate embodiments, the phenotype may be an in-cell reporter assay. Non-limiting examples of suitable in-cell reports assay include in-cell ELISA (also known as in-cell Western) and in-cell PCR (or RT-PCR). In-cell reporter assays permit the visualization of a molecular event that occurs during oocyte maturation or early embryonic development. In many instances, a molecular event precedes a cellular event. For example, the phosphorylation (i.e., activation) of MAPK occurs well in advance of GVBD. The molecular event can be visualized through the use of labeled secondary antibodies or labeled PCR probes. Suitable labels include, but are not limited to, fluorescent, luminescent, infrared (IR), near-IR, ultraviolet (uv), radioactive, and colorimetric (e.g., a colorless or soluble substrate is converted to a colored or precipitated product).

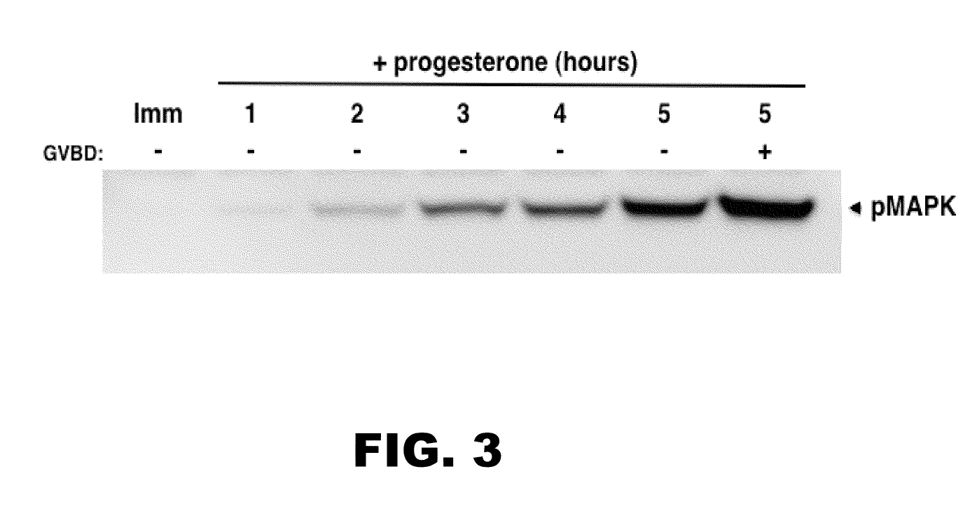

In some instances, in-cell ELISA assays can be used to detect proteins synthesized from maternally-inherited mRNAs (e.g., Musashi, CPEB, Ringo, Mos, Cyclin A1, Cyclin B1, Cyclin B2, Cyclin B5, CDK1, Wee1, Cdc25, etc.) and/or zygotically-produced mRNAs (e.g., activin, Vg1, VegT, β-catenin, Xwnt8, Frzb, TGF-β, BMPs, Hedgehog, Goosecoid, Chordin, Xlin 1, Xnot, Nodal-related, Xbra, HOXs, etc.). The proteins of interest can be detected by using specific primary antibodies (moreover, more than one protein can be detected simultaneously via the use of more than one primary antibody and differentially-labeled secondary antibodies). In-cell ELISA assays also can be used to detect activation of signaling pathways (e.g., MAPK, Wnt, Notch, Hedgehog, TGF-β, Ras, IP₃, GSK3, GTPases, RTKs, Jak/STAT, etc.) via the use of phospho-specific or other activation-specific antibodies. For an example, the activation and phosphorylation of MAP kinase can be detected during oocyte maturation (and prior to GVBD) using phospho-specific MAPK antibodies (see FIG. 3). Similarly, activation of MPF (a complex of CDK1 and Cyclin B) can be detected during meiotic and mitotic cell cycles using phospho-specific antibodies (i.e., loss of inhibitory phosphorylation on CDK1 is indicative of activation). In-cell RT-PCR assays can be used to monitor the presence of specific transcripts and/or the relative level of a specific transcript. The transcripts may be maternally-inherited or zygotically-produced. In other embodiments, pools of oocytes or embryos may be removed at specified intervals and RT-PCR performed in vitro to allow detection of multiple distinct transcripts simultaneously using primers that generate different sized products as necessary.

The molecular and/or cellular events detected by the reporter-based assays and/or the in-cell reporter assays may be stimulated or inhibited by the compounds of interest.

(ii) Monitoring Means

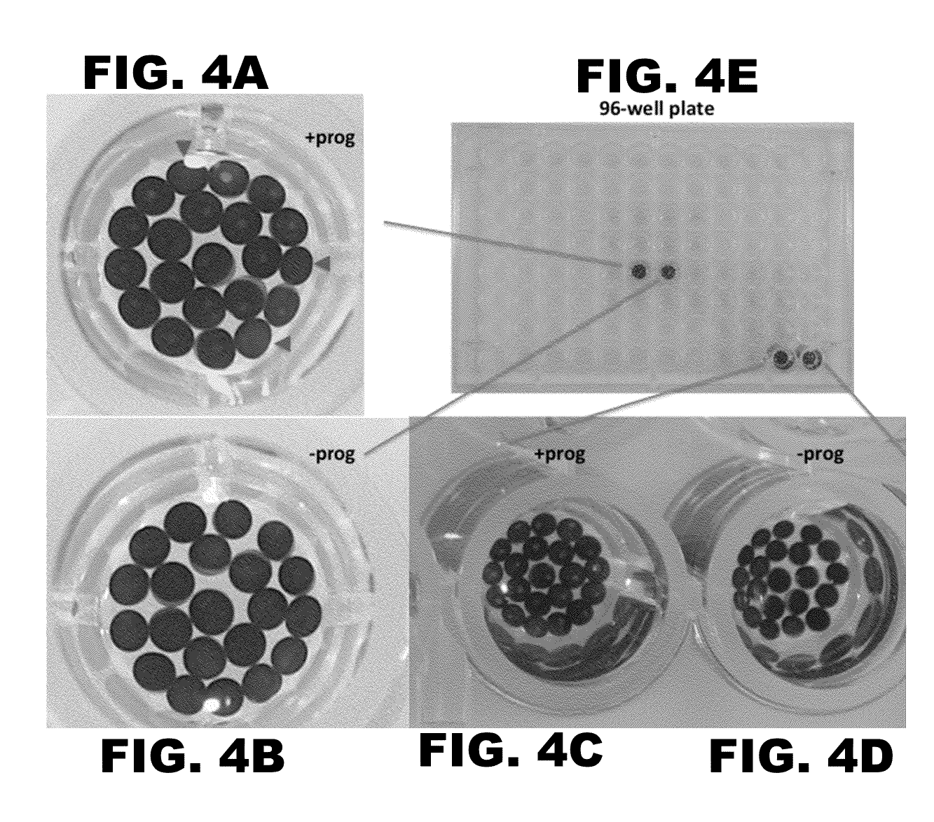

The phenotype may be monitored visually by microscope or by using a variety of detection/image capture devices. Non-limiting examples of suitable detection/image capture devices include cameras, imaging systems, plate readers, or camera-mounted microscope systems. The detection/image capture device may utilize visible light, fluorescent light, IR light, or uv light. The light may illuminate the multi-well system from the top, bottom, or side. The detection/image capture device may be coupled to image processing systems, image analysis systems (e.g., systems that automatically score for the desired phenotype from the captured images), and/or digital storage systems. In exemplary embodiments, the detection/image capture system is capable of taking high resolution images that can be digitally zoomed to analyze subregions of the image. For example, a high content, high resolution image of a multi-well system can be digitally zoomed to analyze oocytes or embryos in individual wells of the system (see FIG. 4).

In some embodiments, the monitoring step may comprise time-lapse image capture. For example, high content digital images may be taken at regular intervals, e.g., intervals of 2, 5, 10, 15, 20, 25, 30, 45, or 60 minutes. The total duration of time-lapse image capture will depend upon the phenotype that is monitored. In general, time-lapse image capture is suitable when the phenotype is GVBD or cell cleavage. Additionally, more than one multi-well system can be concurrently monitored by placing the multi-well systems on a rotating stage or platform. For example, two, three, four, five, or more multi-well systems may be placed on the rotating stage/platform, wherein the stage/platform can be automatically rotated and images can be automatically acquired at regular intervals.

In other embodiments, the monitoring step may comprise end-point image capture. Typically, in-cell reporter assays are performed in fixed oocytes or embryos. For example, an in-cell ELISA comprises fixation, permeabilization, incubation with primary antibodies, incubation with secondary antibodies, and a final clearing step for whole-mount visualization of the oocytes or embryos. In some embodiments, the in-cell reporter phenotype may be monitored at a single time point. The single time point generally will vary depending upon the molecular event that is monitored. For example, phosphorylation (i.e., activation) of MAPK may be monitored at about 1-4 hours after hormone stimulation. In other embodiments, the in-cell reporter phenotype may be monitored at multiple time points by removing subsets of oocytes or embryos from each reaction well at the appropriate time points and subjecting the subsets to the in-cell reporter assay. Thus, multiple end points of a molecular event detected with an in-cell reporter assay may be monitored.

### (c) Identifying Molecular Mechanisms

The method may further comprise the step of determining a molecular mechanism of action for the compound of interest. Means for deciphering the mechanism of action of a compound are well known in the art. Suitable biochemical, molecular, or cellular assays include, without limit, Western blot assays, ELISA assays, PCR-based assays, enzyme assays, phosphorylation assays, cell cycle assays, protein-protein interaction assays, RNA-protein interaction assays, RNA polyadenylation assays, protein synthesis assays, ligand binding assays, receptor binding assays, immunoprecipitation assays, kinetic assays, immunohistochemical localization assays, and the isolation and characterization of complexes comprising a tagged (e.g., biotin-labeled) compound and a cellular target (e.g., protein).

### DEFINITIONS

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, “phenotype” refers to a trait or feature than can be visually observed in oocytes or embryos. In some instances, the phenotype is a morphological trait such as germinal vesicle breakdown (GVBD) or embryonic cleavage divisions. In other instances, the phenotype is an in-cell reporter assay in which a molecular event is visualized through the use of reporter molecules such as labeled antibodies or probes.

Having described the invention in detail, it will be apparent that modifications and variations are possible without departing from the scope of the invention defined in the appended claims.

### EXAMPLES

### Example 1

### High Definition Timed Imaging of Oocyte Maturation

Oocyte maturation was monitored at regular intervals using a high content image capture procedure. Immature stage VI Xenopus oocytes were transferred to wells of a flat bottom 96-well plate containing about 178 μL of L15 culture media. The oocytes were transferred using wide mouth 200 μL pipette tips with minimal transfer of culture medium (e.g., about 20 μL). Generally, the oocytes oriented with the dark-pigmented animal hemisphere facing up due to the higher density of the yolk-containing vegetal hemisphere. Incorrectly orientated oocytes were gently manipulated using a drawn-out capillary tube or other tool to rotate them into the correct orientation. It was found that 21 oocytes per well was optimal as they formed a regular 13:7:1 monolayer array that filled the flat bottom of the well and, essentially, locked the oocytes in the correct orientation. The oocytes were incubated for about 16 hours (i.e., overnight) at 16-25° C. in the presence of 1% DMSO (i.e., 2 μL of 100% DMSO).

Oocyte maturation was stimulated by the addition of progesterone. For this, 2 μL of 1000× stock solution (2 mg/mL in ethanol) was added to the appropriate wells to yield a final concentration of 2 μg/mL (although other concentrations of progesterone may be employed). The 96-well plate was imaged from the top-down (see FIG. 4E) using a 36-megapixel Nikon D800 SLR camera with an AF Micro Nikkor 60 mm, f/2.8 lens, mounted on a dedicated copy stand (with adjustable flanking light sources).

The plate was imaged prior to and after addition of progesterone (i.e., images were taken at regular intervals over a period of 7 hours). Oocyte maturation was phenotypically assessed by the appearance of a white spot at the animal pole of the oocyte. The white spot is due to migration of the germinal vesicle towards the animal pole prior to germinal vesicle breakdown (GVBD). The high resolution images were digitally zoomed after capture to analyze the maturation status of oocytes in individual wells of the plate (FIGS. 4A-D). Oocyte GVBD was readily observed in oocytes treated with progesterone (FIGS. 4A and 4C as compared to those not treated with progesterone (FIGS. 4B and 4D) following digital zoom of captured images. Some minor distortion was observed in the most peripheral well images (see FIGS. 4C and 4D), but the entire field of oocytes could nonetheless were easily scored.

### Example 2

### Using the Xenopus Oocyte Bioassay to Screen Test Compounds

The Xenopus oocyte bioassay was used to screen a collection of about 2000 low molecular weight small molecules. Oocytes were distributed among multi-well plates essentially as described above in Example 1. The test compounds were dissolved in DMSO to make 100× stock solutions. The test compounds were added to the appropriate wells at a final concentration of 100 μM (and 1% DMSO). No drug control oocytes were exposed to 1% DMSO only. Sixteen hours later, progesterone was added to the appropriate wells, with the first and last wells of each plate containing the no drug control oocytes. The rate of maturation in the first and last wells served as an intra-assay control for time taken for progesterone addition across each multiwell plate. The plates were imaged essentially as described above in Example 1.

A total of 1952 compounds were screened and 278 compounds were identified that specifically modulated oocyte maturation (14.2% hit rate). Three classes of compounds were identified: activators (113 compounds, 5.8%), which trigger maturation in the absence of progesterone; inhibitors (161 compounds, 8.2%), which delay progesterone-dependent maturation; and accelerators (4 compounds, 0.2%), which accelerate progesterone-stimulated maturation but do not trigger maturation without progesterone.

Approximately 350 parthenolide derivatives were screened and the following modulators of Xenopus oocyte maturation were identified. PNR-5-46, JVM-9, JVM-59, and PNR-81 were identified as activators (i.e., triggered progesterone-independent oocyte maturation). PNR-5-41 was identified as an inhibitor (i.e., attenuated the rate of progesterone-induced oocyte maturation). JVM-20 was found to inhibit only at high dose (100 μM) and to accelerate progesterone-stimulated maturation from 1 to 50 μM.

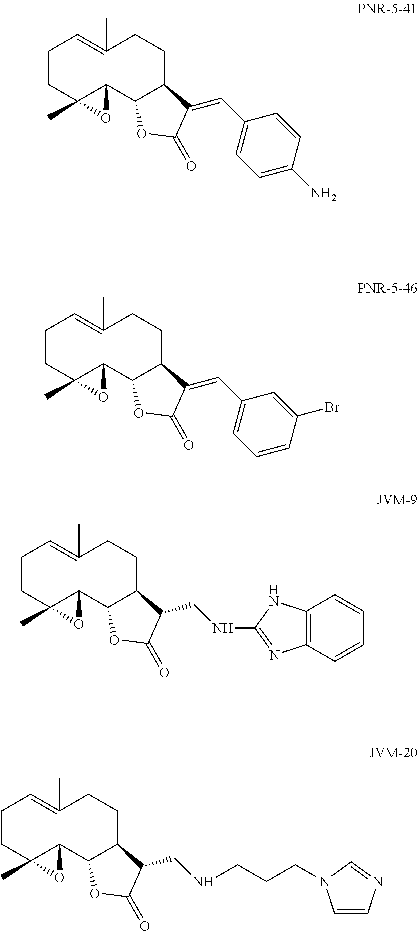 [SMILES: C/C1=C\CC[C@@]2(C)O[C@H]2[C@H]2OC(=O)/C(=C\c3ccc(N)cc3)[C@@H]2CC1 ; C/C1=C\CC[C@@]2(C)O[C@H]2[C@H]2OC(=O)/C(=C\c3cccc(Br)c3)[C@@H]2CC1 ; C/C1=C\CC[C@@]2(C)O[C@H]2[C@H]2OC(=O)[C@@H](CNc3nc4ccccc4[nH]3)[C@@H]2CC1 ; C/C1=C\CC[C@@]2(C)O[C@H]2[C@H]2OC(=O)[C@@H](CNCCCn3ccnc3)[C@@H]2CC1]

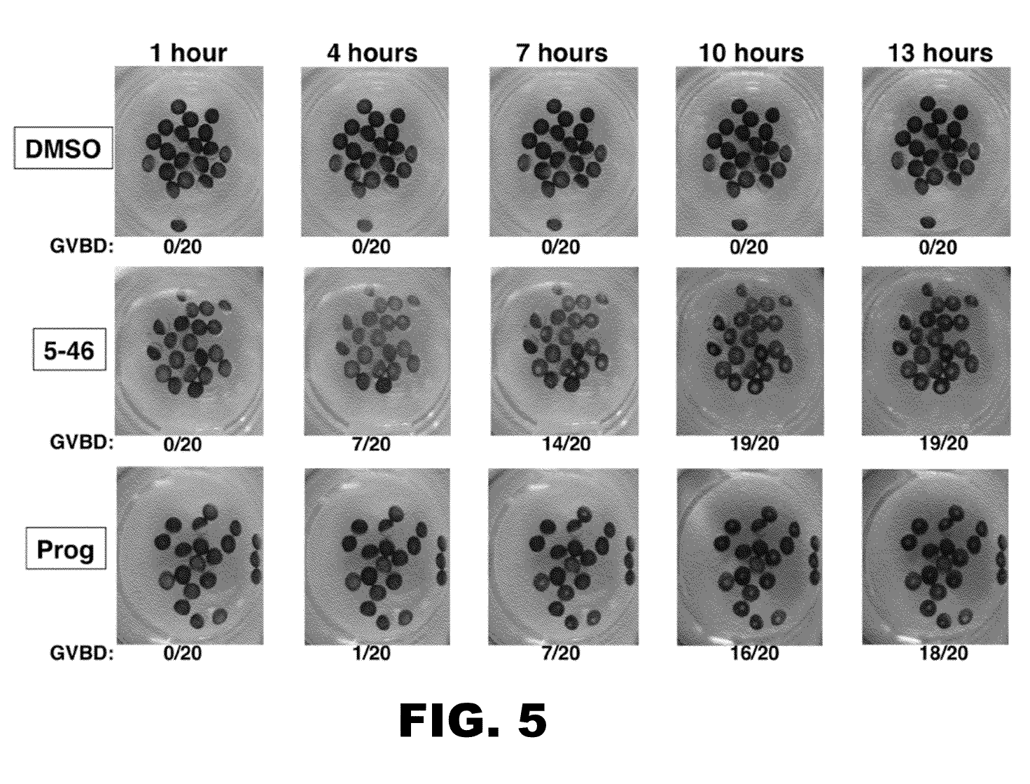

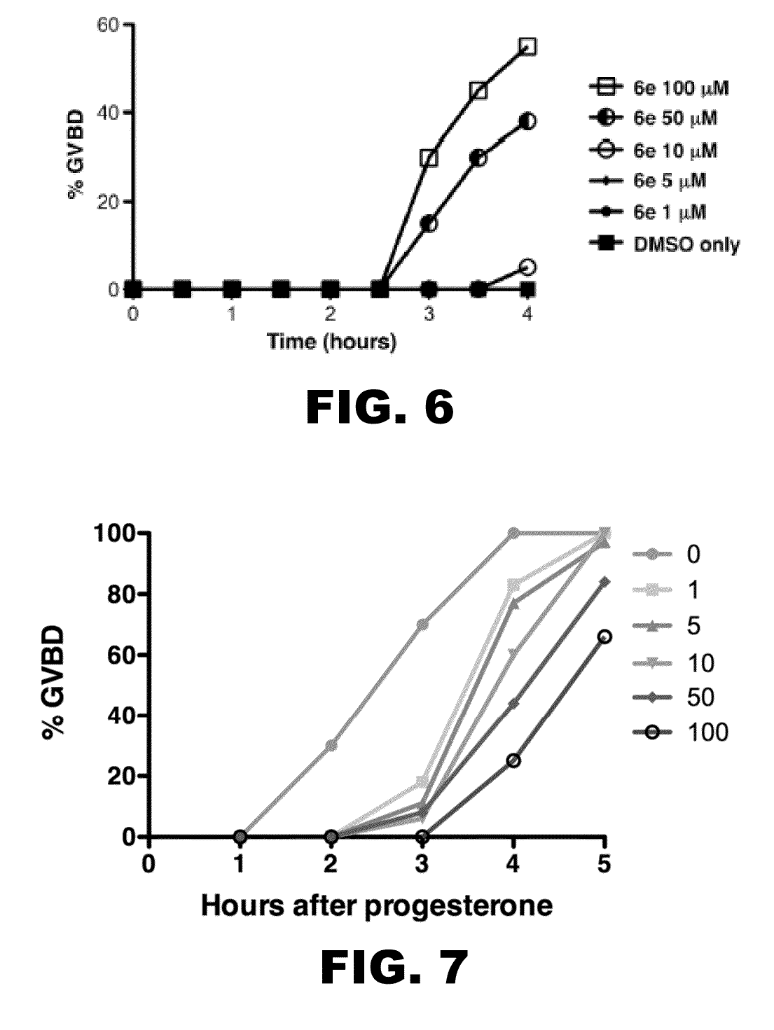

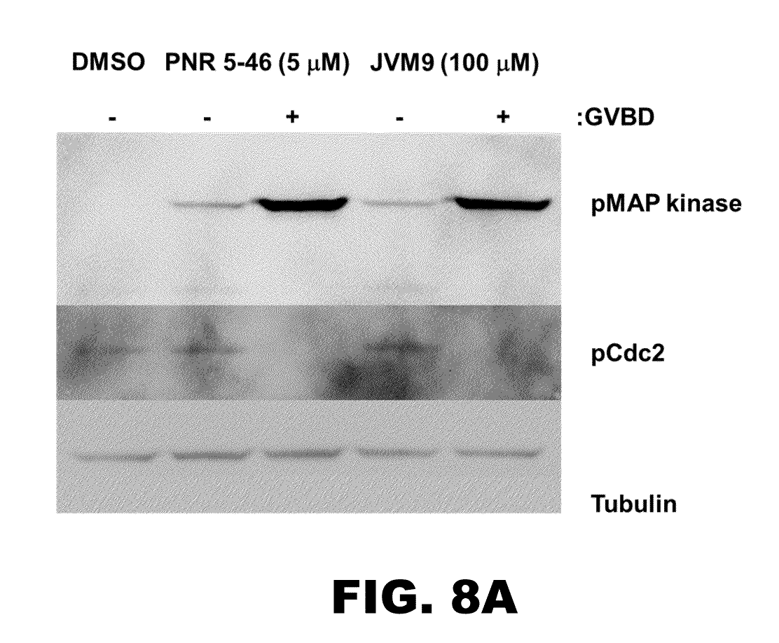

As an example of the efficiency of the screening assay, digital time-lapse images of oocytes treated with DMSO, PNR-5-46, or progesterone are shown in FIG. 5. DMSO-treated oocytes did not undergo GVBD during the 13 hour period. PNR-5-46 induced oocyte maturation in the absence of progesterone. About 35% of the PNR-5-46-treated oocytes initiated GVBD by about 4 hours. In contrast, about 35% of the progesterone-treated oocytes started GVBD by about 7 hours. The EC₅₀ of PNR-5-46 was determined to be 7 μM. JVM-9 also activated spontaneous oocyte maturation; a dose-response curve of JVM-9 is shown in FIG. 6. JVM-9 activated GVBD at concentrations of at 100 and 50 μM, but was less effective at 10 μM. PNR-5-41 inhibited progesterone-induced maturation in a dose-dependent manner (see FIG. 7). The EC₅₀ of PNR-5-41 was determined to be 5 μM, which was significantly different from the DMSO only treatment (p<0.05, n=5).

### Example 3

### Molecular Mechanisms of Action of the Identified Compounds

To begin to decipher the mechanism of action of the compounds that modified oocyte maturation, their effects on regulators of the meiotic cell cycle were examined. In particular, Musashi (Msi1) phosphorylation, Msi2 phosphorylation, MAP kinase phosphorylation, and Cdc2 (also called CDK1) dephosphorylation were examined in Xenopus oocytes treated with a modifying compound or only DMSO (i.e., control). The oocytes were processed for Western blot analysis using standard procedures, and the blots were probed with antibodies specific for phosphorylated or non-phosphorylated forms of the proteins of interest.

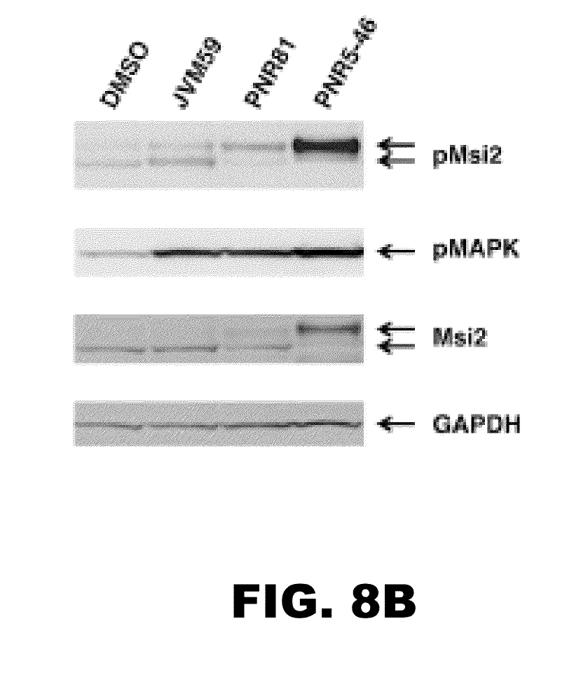

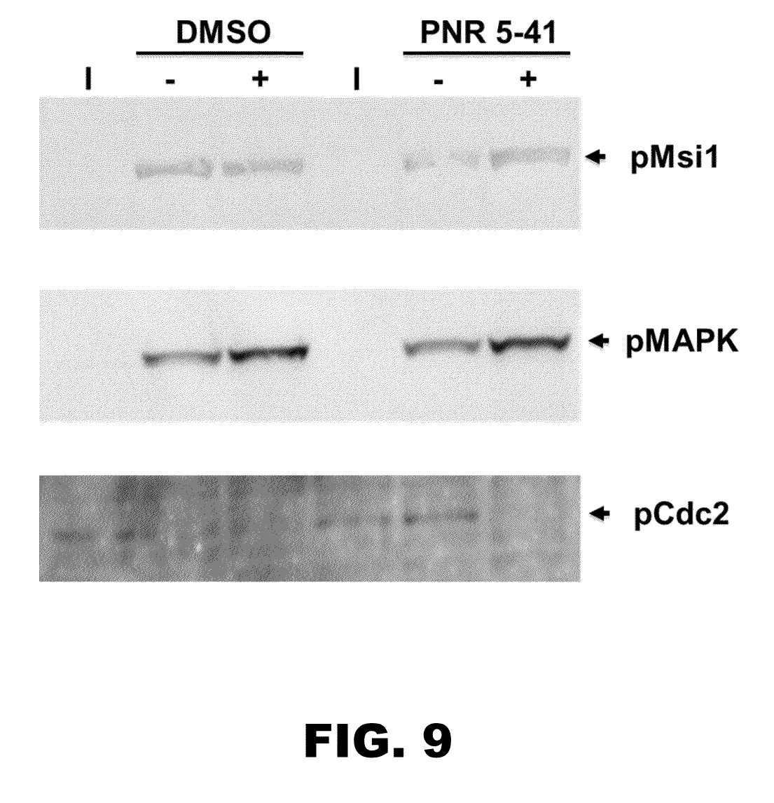

As shown in FIG. 8A, the activator compounds, PNR-5-46 and JVM-9, promoted phosphorylation of MAP kinase and dephosphorylation of cdc2 (also called CDK1), which corresponds to activation of MPF (a complex of cyclin B and CDK1). Additional experiments revealed that PNR-5-46 triggered activation of Msi2 (see FIG. 8B), as well as Msi1 (not shown) using antisera recognizing a conserved site of activating phosphorylation on Musashi required for target mRNA translation. The phosphorylation of Msi2 caused a mobility shift in Msi2 protein. The inhibitor compound, PNR-5-41, did not affect phosphorylation of Msi1 or MAP kinase (which are normally activated in response to progesterone), but de-phosphorylation and activation of cyclin B/CDK1 (i.e., MPF) was blocked, indicating the compound functioned downstream of MAP kinase (see FIG. 9).

### Example 4

### Identified Compounds Inhibit Mammalian Cancer Stem Cell Self-Renewal

Mammosphere culture growth presents a useful indicator of the presence of breast cancer cells with stem cell-like properties. Breast cancer cell lines grown as mammospheres (under non-adherent plating conditions) recapitulate the three-dimensional organization of tumors. Importantly, assessment of stem cell self-renewal capacity can be achieved through dispersion of mammospheres to single cells and subsequent limited dilution replating. To determine whether PNR-5-46, PNR-5-41, or JVM-20 inhibited mammosphere formation in this system, MCF-7 or MDA MB231 cells were cultured lines for 3 days in media containing the test compound (50 μM) or DMSO, and then cultured in the absence of the test compound. At day 7, mammospheres were collected, dispersed to single cells and replated at limiting dilution (P1). Spheroids were scored at day 14 (P2). Mammosphere forming units (MFUs) are defined as spheroid bodies >100 μm (MCF-7 cells) or >65 μm (MDA MB231 cells) diameter after 7 (P1) or 14 (P2) days growth in non-adherent culture.

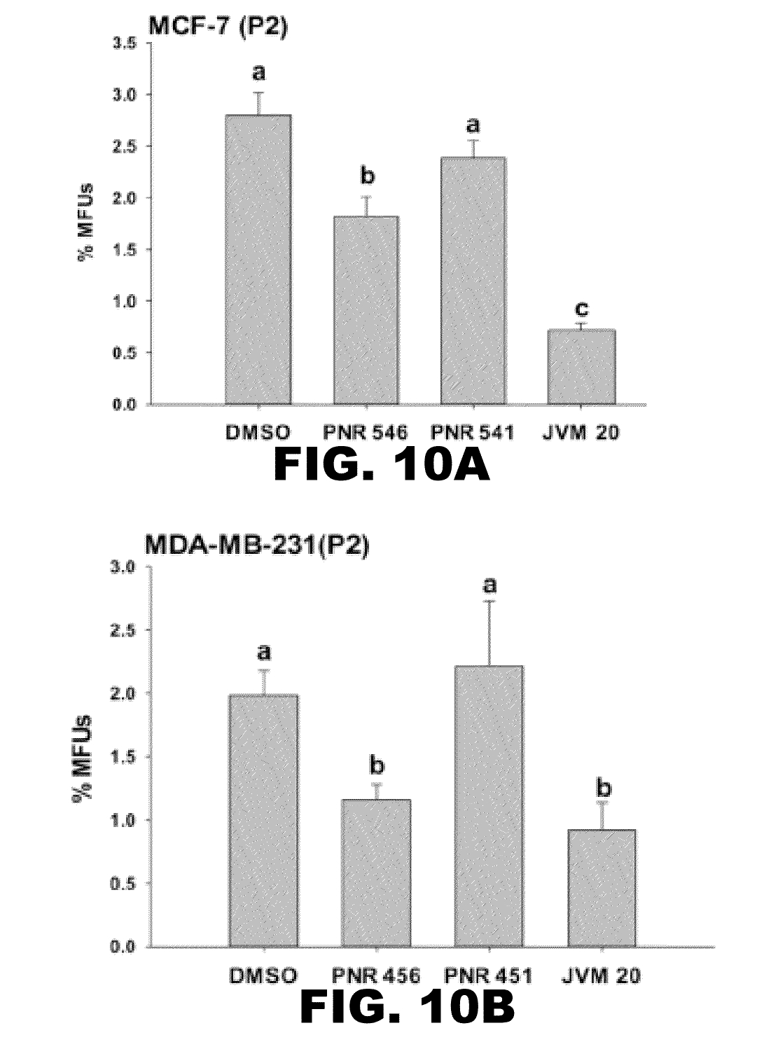

PNR-5-46 and JVM-20 effectively inhibited mammosphere formation of both MCF-7 or MDA MB231 cells relative to DMSO-treated control cells, whereas PNR-5-41 did not (FIGS. 10A,B). This effect was observed at 50, 5, and 0.5 μM doses. Since the compounds were only present during the first three days of culture, inhibition of mammosphere formation on day 14 following the dispersion and re-plating on day 7, reflects a sustained impact on the cancer stem cell population. When assessed at the 0.5 μM dose, there was no significant effect of drug treatment on general cell viability, indicating the PNR-5-46 and JVN-20 specifically targeted cancer stem cell functionality. These findings suggest that molecules that impinge upon mRNA translational control during Xenopus oocyte maturation may be active in the regulation of stem cell self-renewal.

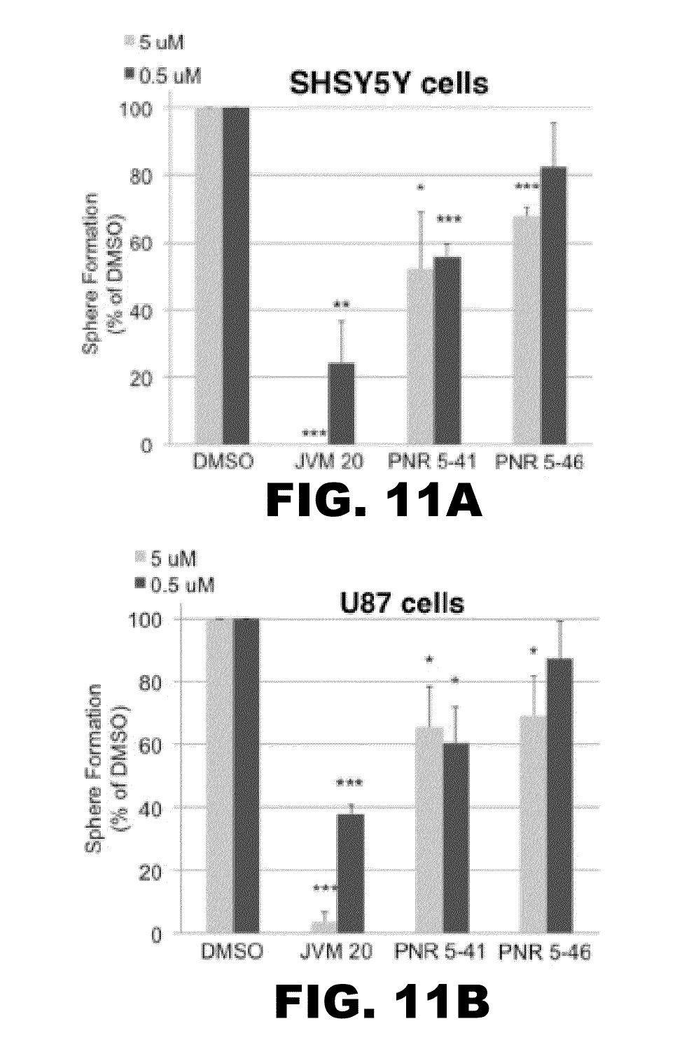

The activity of the three test compounds to inhibit neurosphere formation was also tested in a neuroblastoma cell line SHSY5Y and a glioblastomoa cell line U87. All three compounds attenuated neurosphere formation in both cell lines, to varying degrees of efficacy (FIGS. 11A,B). JVM-20 was particularly effective, even at the lowest dose tested (0.5 μM). None of the compounds affected cell viability. Significant inhibition of SHSY5Y and U87 stem cell self-renewal was also seen for PNR-5-41, but not for PNR-5-46. Interestingly, PNR-5-41 was effective for attenuation of neural cancer stem cell function but was not effective against breast cancer stem cells, whereas PNR-5-46 was effective for breast cancer stem cells but was not as effective against neural cancer stem cell at low dose. Together these data indicate a differential sensitivity of breast and neural cancer stem cells to different parthenolide derivatives.

### Example 5

### Phenotypic Imaging of Embryonic Development

Fertilized one-cell Xenopus embryos undergo 12 rapid, synchronous cleavage divisions, followed by a period of slower, asynchronous cell divisions. The transition to the slower rate of cell division is termed the midblastula transition (MBT) and coincides with the onset of gene transcription. The first cleavage division occurs about 90 min after fertilization and the subsequent rapid divisions occur at about 20-30 min intervals.

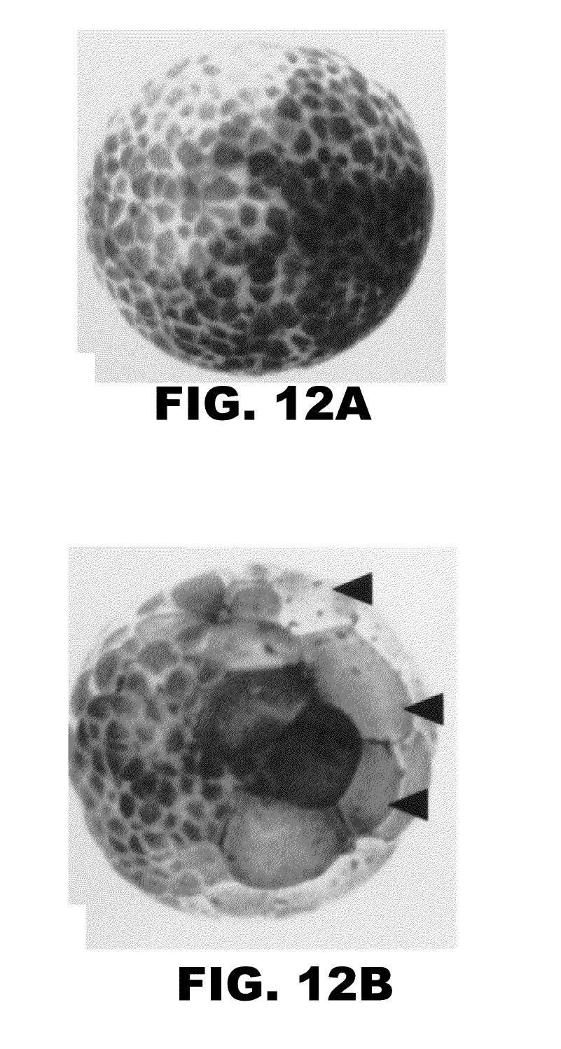

To verify that alterations in the timing or symmetry of the early cleavage divisions can be monitored visually, fertilized one-cell embryos were microinjected with RNA encoding oncogenic Raf-1 (vRaf) to arrest mitotic cell cycle progression. Control one-cell embryos were microinjected with water. The embryos were allowed to develop to the blastula stage. Control embryos contained many uniformly-sized cells (FIG. 12A), whereas vRaf-injected embryos contained several very large arrested cells (FIG. 12B).

The early cell cleavages in Xenopus embryos can be imaged in multi-well plates essentially as described in Example 1. Each well can contain about 3-4 embryos. Embryos may be cultured for up to 14 days to observe developmental consequences arising from culture in test compounds. High definition images can be taken at regular intervals to monitor the early rapid synchronous cleavages in the absence and presence of test compounds (control embryos can be exposed to vehicle (DMSO) only). The molecular mechanism of action of compounds of interest can be determined using Western blotting and other suitable assays.

### Example 6

### In-Cell MAP Kinase Activation Assay

An in-cell ELISA assay will be developed to monitor activation of MAP kinase and activation of MPF in Xenopus oocytes. Activation of MAP kinase can be monitored by detecting the phosphorylation of MAP kinase (e.g., using phospho-MAP kinase 1/2 antibodies from Thermo Scientific) and activation of MPF can be monitored by detecting the dephosphorylation of CDK1 (e.g., using phospho-specific CDK antibodies from Cell Signaling). Oocytes may be distributed among the well of a 96-well plates essentially as described above, except fewer oocytes may be required and no orientation correction will be needed. In-cell ELISA assays may be performed at several time points to determine the optimal times, relative will GVBD50, to asses gain of phospho-MAPK and loss of phospho-CDK. A parallel set of time-matched oocytes may be processed for standard Western blotting to allow verification of activation detected in the in-cell assay relative to the Western signal. To further validate the in-cell ELISA assay, oocytes may be treated with PNR-5-41 to determine whether MAPK activation is attenuated relative to vehicle-treated control embryos. Additionally, oocytes may be treated with PNR-5-46 or JVM-9 to determine whether MAPK activation is accelerated relative to vehicle-treated control embryos.

1. An in vivo method for screening a plurality of compounds, the method comprising:
a) contacting a plurality of amphibian oocytes or embryos with the plurality of compounds; and
b) monitoring a phenotype in the plurality of amphibian oocytes or embryos to identify a compound that affects the phenotype.

2. The method of claim 1, wherein the method is performed in a multi-well format or a high throughput screening format.

3. The method of claim 1, wherein the monitoring step comprises time-lapse digital image capture.

4. The method of claim 1, wherein the phenotype is germinal vesicle breakdown, cell cleavage, a reporter-based assay, or an in-cell reporter assay.

5. The method of claim 4, wherein germinal vesicle breakdown is altered in a hormone-dependent manner or a hormone-independent manner; and cell cleavage is altered temporally, spatially, or both.

6. The method of claim 1, wherein the amphibian oocytes or embryos are from a Xenopus species or a Rana species.

7. The method of claim 1, wherein the amphibian oocytes or embryos are wild type, mutant, or genetically-modified.

8. The method of claim 1, wherein the plurality of compounds is a small molecule library, a pharmaceutically active compound library, a natural product library, a carbohydrate library, a lipid molecule library, a nucleic acid library, an antisense oligonucleotide library, a microRNA library, or a peptide library.

9. The method of claim 1, wherein the method further comprises c) determining a molecular mechanism of action for the compound.

10. The method of claim 1, wherein the plurality of compounds is a library of small molecules; the plurality of amphibian oocytes or embryos is from Xenopus laevis; and the phenotype is germinal vesicle breakdown, cell cleavage, a reporter-based assay, or an in-cell reporter assay.

11. A method for identifying a compound that affects a regulated mRNA translation control process, the method comprises:
a) contacting a plurality of amphibian oocytes or embryos with a plurality of compounds; and
b) monitoring a phenotype in the plurality of amphibian oocytes or embryos to identify the compound that affects the regulated mRNA translation control process.

12. The method of claim 11, wherein the method is performed in a multi-well format or a high throughput screening format.

13. The method of claim 11, wherein the monitoring step comprises time-lapse digital image capture.

14. The method of claim 11, wherein the phenotype is germinal vesicle breakdown, cell cleavage, a reporter-based assay, or an in-cell reporter assay.

15. The method of claim 14, wherein germinal vesicle breakdown is altered in a hormone-dependent manner or a hormone-independent manner; and cell cleavage is altered temporally, spatially, or both.

16. The method of claim 11, wherein the amphibian oocytes or embryos are from a Xenopus species or a Rana species.

17. The method of claim 11, wherein the amphibian oocytes or embryos are wild type, mutant, or genetically-modified.

18. The method of claim 11, wherein the plurality of compounds is a small molecule library, a pharmaceutically active compound library, a natural product library, a nucleic acid library, an antisense oligonucleotide library, a microRNA library, or a peptide library.

19. The method of claim 11, wherein the regulated mRNA translation process comprises a control protein chosen from Pumilio1, Pumilio2, Musashi1, Musashi2, or the cytoplasmic polyadenylation element binding protein (CPEB).

20. The method of claim 11, wherein the method further comprises c) determining a molecular mechanism of action for the compound.

21. (canceled)
